(19) United States
(12) Patent Application Publication
(10) Pub. No.: US 2006/0013852 A1
(43) Pub. Date: Jan. 19, 2006

(54) Use of organic compounds

(76) Inventor: Margaret Forney Prescott, Millburn, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/519,070

(22) PCT Filed: Jun. 27, 2003
(86) PCT No.: PCT/EP03/06855

§ 371 Date: Aug. 10, 2005

Related U.S. Application Data
(60) Provisional application No. 60/392,586, filed on Jun. 28, 2002.

(51) Int. Cl. A61K 31/445; A61K 31/4184; A61K 31/165

(52) U.S. Cl. 424/423; 514/317; 514/381; 514/616

(57) ABSTRACT

The present invention relates to drug delivery systems, comprising an ARB or a RI, or at least two representatives selected from the group consisting of an ARB, an ACEI and a RI, or, in each case, a pharmaceutically acceptable salt thereof, for the prevention and treatment of proliferative diseases, particularly vascular diseases. The invention furthermore relates to the use of such drug delivery systems, for preventing or treating restenosis in diabetic and non-diabetic patients, or for the prevention or reduction of vascular access dysfunction in association with the insertion or repair of an indwelling shunt, fistula or catheter in a subject in need thereof.

The present invention relates to drug delivery systems, comprising an angiotensin II antagonist (ARB) or a renin inhibitor (RI), or at least two representatives selected from the group consisting of an ARB, an angiotensin converting enzyme inhibitor (ACE-inhibitor) and a RI, or, in each case, a pharmaceutically acceptable salt thereof, for the prevention and treatment of proliferative diseases, particularly vascular diseases. The invention furthermore relates to the use of such drug delivery systems, for preventing or treating restenosis in diabetic and non-diabetic patients, or for the prevention or reduction of vascular access dysfunction in association with the insertion or repair of an indwelling shunt, fistula or catheter in a subject in need thereof.

Many humans suffer from circulatory diseases caused by a progressive blockage of the blood vessels that perfuse major organs such as heart, liver and kidney. Severe blockage of blood vessels in such humans often leads to e.g. ischemic injury, hypertension, stroke or myocardial infarction. Atherosclerotic lesions, which limit or obstruct coronary or peripheral blood flow are the major cause of ischemic disease related morbidity and mortality including coronary heart disease, stroke, aneurysm and peripheral claudication. To stop the disease process and prevent the more advanced disease states in which the cardiac muscle or other organs are compromised, medical revascularization procedures such as percutaneous transluminal coronary angioplasty (PCTA), percutaneous transluminal angioplasty (PTA), stenting, atherectomy, bypass grafting or other types of vascular grafting procedures are used. A similar growth into the vessel lumen and obstruction of blood flow occurs within bypass grafts, at sites of anastomoses in transplantion and in vessels used to create dialysis access, thus revascularization procedures such angioplasty and/or stenting are also used in these pathologic conditions.

Complications associated with vascular access devices is a major cause of morbidity in many disease states. For example, vascular access dysfunction in hemodialysis patients is generally caused by outflow stenoses in the venous circulation. Vascular access related morbidity accounts for about 23 percent of all hospital stays for advanced renal disease patients and contributes to as much as half of all hospitalization costs for such patients. Additionally, vascular access dysfunction in chemotherapy patients is generally caused by outflow stenoses in the venous circulation and results in a decreased ability to administer medications to cancer patients. Often the outflow stenoses is so severe as to require intervention. Additionally, vascular access dysfunction in total parenteral nutrition (TPN) patients is generally caused by outflow stenoses in the venous circulation and results in reduced ability to care for these patients. Up to the present time, there has not been any effective drug for the prevention or reduction of vascular access dysfunction that accompany the insertion or repair of an indwelling shunt, fistula or catheter, such as a large bore catheter, into a vein in a mammal, particularly a human patient. Survival of patients with chronic renal failure depends on optimal regular performance of dialysis. If this is not possible (for example as a result of vascular access dysfunction or failure), it leads to rapid clinical deterioration and unless the situation is remedied, these patients will die. Hemodialysis requires access to the circulation. The ideal form of hemodialysis vascular access should allow repeated access to the circulation, provide high blood flow rates, and be associated with minimal complications. At present, the three forms of vascular access are native arteriovenous fistulas (AVF), synthetic grafts, and central venous catheters. Grafts are most commonly composed of polytetrafluoroethylene (PTFE, or Gore-Tex). Each type of access has its own advantages and disadvantages.

Vascular access dysfunction is the most important cause of morbidity and hospitalization in the hemodialysis population. Venous neointimal hyperplasia characterized by stenosis and subsequent thrombosis accounts for the overwhelming majority of pathology resulting in dialysis graft failure.

The most common form of vascular access procedure performed in chronic hemodialysis patients in the United States is the arteriovenous polytetrafluoroethylene (PTFE) graft, which accounts for approximately 70% of all hemodialysis access.

It has been previously shown that venous neointimal hyperplasia (VNH) in the setting of arteriovenous hemodialysis grafts is characterized by proliferation of smooth muscle cells, and the abundance of neointimal and adventitial microvessels and extracellular matrix components. However, despite a reasonable knowledge of the pathology of VNH, there are still no effective interventions for either the prevention or treatment of hemodialysis vascular access dysfunction. This is particularly unfortunate, as VNH in the setting of hemodialysis grafts appears to be a far more aggressive lesion as compared to the more common arterial neointimal hyperplasia that occurs in peripheral bypass grafts. Compare the 50% one patency in PTFE dialysis access grafts with an 88% five year patency for aortoiliac grafts and a 70 to 80% one year patency for femoro-popliteal grafts. Venous stenoses in the setting of dialysis access grafts also have a poorer response to angioplasty (40% three month survival if thrombosed and a 50% six month survival if not thrombosed) as compared to arterial stenoses.

Despite the magnitude of the problem and the enormity of the cost, there are currently no effective therapies for the prevention or treatment of venous neointimal hyperplasia in dialysis grafts.

Coronary balloon angioplasty was introduced in the late 1970s as a less invasive method for revascularization of coronary artery disease patients. This has led to a quick progress in the development of new percutaneous devices to treat atherosclerotic vasculopathies. However, the expanded use of angioplasty has shown that the arteries react to angioplasty by both a constrictive and a proliferative process similar to wound healing that limits the success of the treatment modality. This process is known as restenosis. Restenosis is defined as a re-narrowing of the treated segment, which equals or exceeds 50% of the lumen in the adjacent normal segment of the artery. Depending on the patient population studied, the restenosis rates range from 30% to 44% of lesions treated by balloon dilation.

This problem prompted a search for interventional techniques that would minimize the risk of restenosis. Gradually, it became clear that the success of any interventional method must be determined by not only how quickly or dependably it opens the diseased artery, but also how likely it is to trigger a the reaction called restenosis.

Re-narrowing e.g. of an artherosclerotic coronary artery after various revascularization procedures occurs in 10-80% of patients undergoing this treatment, depending on the procedure used as well as the arterial site. Besides opening an artery obstructed by atherosclerosis, revascularization in general, but especially revasculoarization using a stent also injures endothelial cells and smooth muscle cells within the vessel wall, thus initiating a thrombotic and inflammatory response that is followed by a proliferative response. Cell derived growth factors such as platelet derived growth factors, endothelial derived growth factors, smooth muscle-derived growth factors (e.g. PDGF, tissue factor, FGF), as well as cytokines, chemokines and lymphokines released from endothelial cells, infiltrating macrophages, lymphocytes, or leukocytes or released from the smooth muscle cells themselves provoke proliferative and migratory responses in the smooth muscle cells as well as additional inflammatory events and neovascularization within the vessel wall. Proliferation/migration of vascular smooth muscle cells usually begins within one to two days post-injury and, depending on the revascularization procedure used, continues for days, weeks, or even months.

Cells within the original atherosclerotic lesion as well as inflammatory cells that have accumulated at the site of injury and stenting, as well as smooth muscle cells and those within the media migrate, proliferate and/or secrete significant amounts of extracellular matrix proteins. Proliferation, migration and extracellular matrix synthesis continue until the damaged endothelial layer is repaired at which time proliferation slows within the intima. The newly formed tissue is called neointima, intimal thickening or restenotic lesion and usually results in narrowing of the vessel lumen. Further lumen narrowing may take place due to constructive remodeling, e.g. vascular remodeling, leading to further loss of lumen size.

A major category of interventional devices called stents has been introduced with the aim of reducing the restenosis rate of balloon angioplasty.

Clinical studies have shown a reduction in the restenosis rates with these endovascular stents. The purpose of stenting is to maintain the arterial lumen by a scaffolding process that provides radial support. Stents, usually made of stainless steel or of a synthetic material, are placed in the artery either by a self-expanding mechanism or, more commonly, using balloon expansion. Stenting results in the largest lumen possible and expands the artery to the greatest degree possible. Stenting also provides a protective frame to support fragile vessels that have had a pathologic dissection due to the revascularization procedures.

However, restenosis remains a major problem in percutaneous coronary intervention, requiring patients to undergo repeated procedures and surgery. Restenosis is the result of the formation of neointima, a composition of smooth muscle-like cells in a collagen matrix. It has been demonstrated that the implantation of stents as part of the standard angioplasty procedure has improved the acute results of percutaneous coronary revascularization, but in-stent restenosis, as well as stenosis proximal and distal to the stent and the inaccessibility of the lesion site for surgical revasculation limits the long-term success of using stents. The absolute number of in-stent restenotic lesions is increasing with the increasing number of stenting procedures, with the complexity of culprit lesion stented as well as with stenting of ever-smaller sized arteries. Neointima proliferation/growth occurs principally within the stented area or proximal or distal to the stented area within 6 months after stent implantation. Neointima is an accumulation of smooth muslce cells within a proteoglycan matrix that narrows the previously enlarged lumen.

Attempts have been made to orally treat restenosis with several pharmaceutically agents, however, these attempts have failed to inhibit restenosis after coronary interventions. Another approach to cope with the situation is to use local intravascular irradiation (brachytherapy or radioactive stents), but the outcome of clinical trials has been hampered by restenosis and/or constrictive remodelling at the edges of the radioactive stents, resulting in an effect called “candy wrapper”.

A recent successful development in the stent device area is the use of stents that release or elute pharmacological agents having antiproliferative and/or antiinflammatory activity.

Accordingly, there is a need for further effective approaches for treatments and the use of drug delivery systems (especially controlled delivery from a catheter-based device (e.g. stents, indwelling shunt, fistula or catheter) or an intraluminal medical device) for preventing and treating intimal thickening or restenosis that occurs after injury due to stenting, e.g. vascular injury, including e.g. surgical injury, e.g. revascularization-induced injury, e.g. anastomotic sites for heart or other sites of organ transplantation, e.g. dialysis access grafts or e.g. anastomoses used to create dialysis access.

Suitable pharmaceutical drugs that can be used for coating stents for local treatment are angiotensin II antagonists (ARBS) and renin inhibitor (RIs), in each case, in free form or in form of a pharmaceutically acceptable salt have beneficial effects when locally applied to the lesions sites. ARBs and RIs are surprisingly well adapted for delivery especially controlled delivery from a catheter-based device or an intraluminal medical device. These pharmaceutical drugs and combinations are particularly stable in any pharmaceutically acceptable polymers at body temperature and in human plasma, permitting an unexpected long storage in coated stents, indwelling shunt, fistula or catheter. They are particularly well adapted because they are easily secured onto the medical device by the polymer and the rate at which they are released from coating to the body tissue can be easily controlled.

Furthermore, our herein described coated stents, indwelling shunt, fistula or catheter permit long-term delivery of the drug(s). It is particularly worthwhile to control the bioeffectiveness of our coated stents, indwelling shunt, fistula or catheter in order to obtain the same biological effect as a liquid dosage.

An advantage of using ARBs and RIs as coating material for stents is that a corresponding drug is applied to the vessel at the precise site and at the time of vessel injury. This kind of local drug administration can be used to achieve higher tissue concentrations of the drug without the risk of systemic toxicity.

The class of angiotensin II receptor antagonists (ARBs) comprises compounds having differing structural features, essentially preferred are the non-peptidic ones. For example, mention may be made of the compounds that are selected from the group consisting of valsartan (cf. EP 443983), losartan (cf. EP253310), candesartan (cf. 459136), eprosartan (cf. EP 403159), irbesartan (cf. EP454511), olmesartan (cf. EP 503785), tasosartan (cf. EP539086), telmisartan (cf. EP 522314), the compound with the designation E-1477 of the following formula 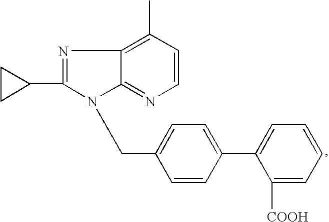
the compound with the designation SC-52458 of the following formula 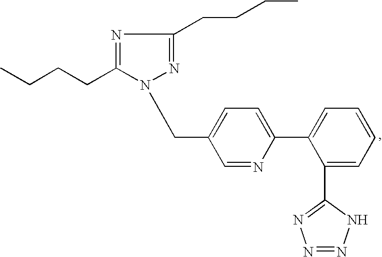
and the compound with the designation the compound ZD-8731 of the following formula 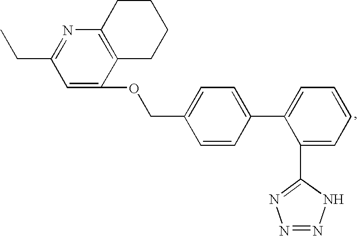
or, in each case, a pharmaceutically acceptable salt thereof.

Preferred ARBs are those agents that have been marketed, most preferred is valsartan or a pharmaceutically acceptable salt thereof.

The interruption of the enzymatic degradation of angiotensin I to angiotensin II with so-called ACE-inhibitors (also called angiotensin converting enzyme inhibitors) is a successful variant for the regulation of blood pressure and also a therapeutic method for the treatment of congestive heart failure.

The class of ACE inhibitors comprises compounds having differing structural features. For example, mention may be made of the compounds which are selected from the group consisting alacepril, benazepril, benazeprilat, captopril, ceronapril, cilazapril, delapril, enalapril, enalaprilat, fosinopril, imidapril, lisinopril, moexipril, moveltopril, perindopril, quinapril, quinaprilat, ramipril, ramiprilat, spirapril, temocapril, trandolapril and zofenopril, or, in each case, a pharmaceutically acceptable salt thereof.

Preferred ACE inhibitors are those agents that have been marketed, most preferred are benazepril, enalapril, lisinopril or ramipril, or, in each case, independently of one another, a pharmaceutically acceptable salt thereof.

Renin inhibitors inhibit the action of the natural enzyme renin. The latter passes from the kidneys into the blood where it effects the cleavage of angiotensinogen, releasing the decapeptide angiotensin I which is then cleaved in the lungs, the kidneys and other organs to form the octapeptide angiotensin II. The octapeptide increases blood pressure both directly by arterial vasoconstriction and indirectly by liberating from the adrenal glands the sodium-ion-retaining hormone aldosterone, accompanied by an increase in extracellular fluid volume. That increase can be attributed to the action of angiotensin II. Inhibitors of the enzymatic activity of renin bring about a reduction in the formation of angiotensin I. As a result a smaller amount of angiotensin II is produced. The reduced concentration of that active peptide hormone is the direct cause of e.g. the antihypertensive effect of renin inhibitors. Accordingly, renin inhibitors or salts thereof can be employed e.g. as antihypertensives or for treating congestive heart failure.

The class of renin inhibitors comprises compounds having differing structural features. For example, mention may be made of compounds which are selected from the group consisting of ditekiren (chemical name: [1 S-[1 R*,2R*,4R*(1 R*,2R*)]]-1-[(1,1-dimethylethoxy)carbonyl]-L-proly I-L-phenylalanyl-N-[2-hydroxy-5-methyl-1-(2-methylpropyl)-4-[[[2-methyl-1-[[(2-pyridinylmethyl)amino]carbonyl]butyl]amino]carbonyl]hexyl]-N-alfa-methyl-L-histidinamide); terlakiren (chemical name: [R-(R*,S*)]-N-(4-morpholinylcarbonyl)-L-phenylalanyl-N-[1-(cyclohexylmethyl)-2-hydroxy-3-(1-methylethoxy)-3-oxopropyl]-S-methyl-L-cysteineamide); zankiren (chemical name: [1S-[1R*[R*(R*)],2S*,3R*]]-N-[1-(cyclohexylmethyl)-2,3-dihydroxy-5-methylhexyl]-alfa-[[2-[[(4-methyl-1-piperazinyl)sulfonyl]methyl]-1-oxo-3-phenylpropyl]amino]-4-thiazolepropanamide), especially the hydrochloride thereof; RO 66-1132 and RO-66-1168 of formulae 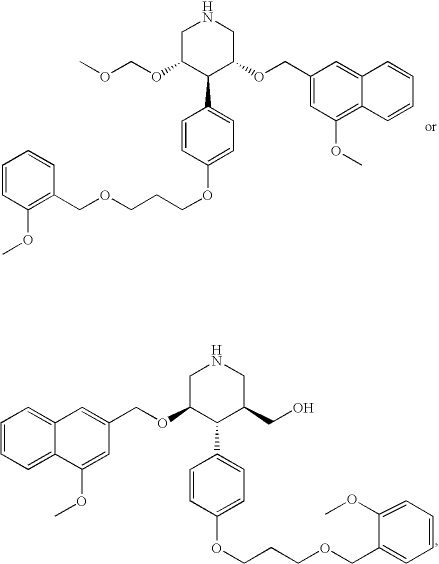
respectively.

Especially preferred is the compound of formula 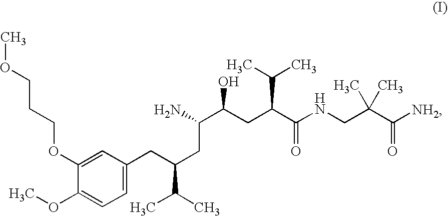
chemically defined as 2(S),4(S),5(S),7(S)-N-(3-amino-2,2-dimethyl-3-oxopropyl)-2,7-di(1-methylethyl)-4-hydroxy-5-amino-8-[4-methoxy-3-(3-methoxy-propoxy)phenyl]-octanamide (generic name: aliskiren), specifically disclosed in EP 678503 A, or a pharmaceutically acceptable salt, especially the hemi-fumarate, thereof.

According to the invention, an ARB or a RI may be applied as the sole active ingredient or in conjunction with each other.

A preferred ARB is valsartan, a preferred RI is aliskiren. In a preferred embodiment they are in conjunction with each other.

The present invention relates to a drug-eluting stent for local treatment, e.g. a stent that elutes or is coated with a coating material or impregnated with a material comprising an ARB or an RI or a mixture of at least two representatives selected from the group consisting of an ARB, an ACEI and an RI, or, in each case, independently of one another, a pharmaceutically acceptable salt thereof.

The present invention relates preferably to a drug-eluting or drug-releasing stent, a drug-delivery vehicle, or a drug delivery device or system comprising an RI or a pharmaceutically acceptable salt thereof.

A preferred ARB is valsartan, preferred RI is aliskiren, a preferred ACEI is benazepril. In a preferred embodiment they are in conjunction with each other.

The present invention relates preferably to a drug-eluting or drug-releasing stent, a drug-delivery vehicle, or a drug delivery device or system comprising at least two representatives selected from the group consisting of valsartan, benazepril, aliskiren, or, in each case, a pharmaceutically acceptable salt thereof.

Preferred combinations comprise valsartan and aliskiren, or valsartan and benazepril, or aliskiren and benazepril or valsartan and benazepril and aliskiren or, in each case, independently of one another, a pharmaceutically acceptable salt thereof.

Most preferred combination comprises aliskiren and benazepril.

Preferably the combination contains between 30 and 70% of aliskiren, between 30 and 70% of valsartan or between 30 and 70% of benazepril.

A preferred triple combination contains between 20 and 40% of aliskiren, between 20 and 40% of benazepril and between 20 and 40% of valsartan.

An appropriate stent to be used according to the invention is a commercially available one, especially a drug that has been approved by health authorities, e.g. the Food and Drug Administration in the USA. Corresponding stent comprise those that uses the balloon-expansion and the self-expansion principles, which can especially have a tubular, ring, multi-design, coil or mesh design. Likewise preferred are biodegragable and biocompatible stents. Suitable stent materials comprise e.g. metals, metal-alloys or polymers having a surface that can be coated. By “biocompatible” is meant a material which elicits no or minimal negative tissue reaction including e.g. thrombus formation and/or inflammation.

A corresponding coating system according to the present invention should be suitable to be used as vehicles for local drug delivery. An appropriate delivery vehicle is to be used that allows the release a predictable and controllable concentration. A delivery vehicle according to the present invention must ensure a controlled release within a time span to be defined by a person skilled in the art and must be suitable for sterilisation.

Drug delivery vehicles comprise a pharmaceutically acceptable polymer selected from the group consisting of polyvinyl pyrrolidone/cellulose esters, polyvinyl pyrrolidone/polyurethane, polymethylidene maloeate, polyactide/glycoloide co-polymers, polyethylene glycol co-polymers, polyethylene vinyl alcohol, and polydimethylsiloxane (silicone rubber).

Examples of polymeric materials include biocompatible degradable materials, e.g. lactone-based polyesters or copolyesters, e.g. polylactide; polylactide-glycolide; polycaprolactone-glycolide; polyorthoesters; polyanhydrides; polyaminoacids; polysaccharides; polyphosphazenes; poly(ether-ester) copolymers, e.g. PEO-PLLA, or mixtures thereof; and biocompatible non-degrading materials, e.g. polydimethylsiloxane; poly(ethylene-vinylacetate); acrylate based polymers or coplymers, e.g. polybutylmethacrylate, poly(hydroxyethyl methylmethacrylate); polyvinyl pyrrolidinone; fluorinated polymers such as polytetrafluoethylene; cellulose esters.

When a polymeric matrix is used, it may comprise 2 layers, e.g. a base layer in which the drug(s) is/are incorporated, e.g. ethylene-co-vinylacetate and polybutylmethacrylate, and a top coat, e.g. polybutylmethacrylate, which is drug(s)-free and acts as a diffusion-control of the drug(s). Alternatively, the drug may be comprised in the base layer and the adjunct may be incorporated in the outlayer, or vice versa. Total thickness of the polymeric matrix may be from about 1 to 500μ, preferably 1 to 20μ or greater. The amount of a drug to be used according to the present invention is about 1 μg to about 500 μg, preferably 10 μg to about 200 μg, per stent. Alternatively, the surface of a stent is loaded with about 1 μg to about 250 μg, preferably about 10 μg to 150 μg, per square centimeter of a compound to be used according the present invention.

The pharmaceutically acceptable polymers do not alter or adversely impact the therapeutic properties of an ARB, an ACEI and a RI. On the contrary, ARBs, ACEIs and RIs are particularly stable in any pharmaceutically acceptable polymers at body temperature and in human plasma, permitting an unexpected long storage in a coated stents.

ARBs, ACEIs and RIs are particularly well adapted because it is easily secured onto the medical device by the polymer and the rate at which it is released from coating to the body tissue can be easily controlled. Furthermore, stents coated with an ARB and a RI permit long-term delivery of the drug. It is particularly worthwhile to control the bioeffectiveness of stents coated with an ARB and a RI in order to obtain the same biological effect as a liquid dosage.

The invention relates to drug-containing delivery systems for the prevention and treatment of proliferative diseases, particularly vascular diseases.

It is also an object of this invention to provide a drug-containing medical device, which allows sustained delivery of the pharmaceutical or sufficient pharmaceutical activity at or near the coated surfaces of the devices.

All the herein mentioned preferences apply to the stents or medical devices e.g. indwelling shunt, fistula or catheter.

Also, it is an object of the invention to provide medical devices with stabilized complexed drug coatings or other methods of drug elution and methods for making such devices.

Additionally, it is an object of the invention to provide a drug-releasing stent or medical devices to allow the timed or prolonged application of the drug to body tissue. It is a further object of the invention to provide methods for making a drug-releasing medical device, which permit timed-delivery or long-term delivery of a drug. Thus, there is a need for improved biocompatible complexed drug coatings, which enhance the biostability, abrasion-resistance, lubricating characteristics and bio-activity of the surface of implantable medical devices, especially complexed drug coatings, which contain heat-sensitive biomolecules. In particular, there is a need for improved, cost efficient complexed drug coatings and devices, which have antithrombogenic and/or anti-restenosis and/or anti-inflammatory properties and for more efficient methods of providing the same. The present invention is directed to meeting these and other needs.

A drug delivery device or system comprising a) a medical device adapted for local application or administration in hollow tubes, e.g. a catheter-based delivery device or intraluminal medical device, and b) a therapeutic dosage of an ARB or an RI, or at least two representatives selected from the group consisting of an ARB, an ACEI and an RI, or, in each case, a pharmaceutically acceptable salt thereof, each being releasably affixed to the catheter-based delivery device or medical device.

Such a local delivery device or system can be used to reduce stenosis or restenosis as an adjunct to revascularization, bypass or grafting procedures performed in any vascular location including coronary arteries, carotid arteries, renal arteries, peripheral arteries, cerebral arteries or any other arterial or venous location, to reduce anastomic stenosis such as in the case of arterial-venous dialysis access with or without polytetrafluoroethylene grafting and with or without stenting, or in in conjunction with any other heart or transplantation procedures, or congenital vascular interventions.

The local administration preferably takes place at or near the vascular lesions sites.

Local administration or application may reduce the risk of remote or systemic toxicity. Preferably the smooth muscle cell proliferation or migration is inhibited or reduced according to the invention immediately proximal or distal to the locally treated or stented area.

The administration may be by one or more of the following routes: via catheter or other intravascular delivery system, intranasally, intrabronchially, interperitoneally or eosophagal. Hollow tubes include circulatory system vessels such as blood vessels (arteries or veins), tissue lumen, lymphatic pathways, digestive tract including alimentary canal, respiratory tract, excretory system tubes, reproductive system tubes and ducts, body cavity tubes, etc. Local administration or application of the drug(s) affords concentrated delivery of said drug(s), achieving tissue levels in target tissues not otherwise obtainable through other administration route.

Means for local drug(s) delivery to hollow tubes can be by physical delivery of the drug(s) either internally or externally to the hollow tube. Local drug(s) delivery includes catheter delivery systems, local injection devices or systems or indwelling devices. Such devices or systems would include, but not be limited to, stents, coated stents, endolumenal sleeves, stent-grafts, liposomes, controlled release matrices, polymeric endoluminal paving, or other endovascular devices, embolic delivery particles, cell targeting such as affinity based delivery, internal patches around the hollow tube, external patches around the hollow tube, hollow tube cuff, external paving, external stent sleeves, and the like. See, Eccieston et al. (1995) Interventional Cardiology Monitor 1:33-40-41 and Slepian, N.J. (1996) Intervent. Cardiol. 1:103-116, or Regar E, Sianos G, Serruys P W. Stent development and local drug delivery. Br Med Bull 2001, 59:227-48 which disclosures are herein incorporated by reference.

Delivery or application of the drug(s) can occur using stents or sleeves or sheathes. An intraluminal stent composed of or coated with a polymer or other biocompatible materials, e.g. porous ceramic, e.g. nanoporous ceramic, into which the drug(s) has been impregnated or incorporated can be used. Such stents can be biodegradable or can be made of metal or alloy, e.g. Ni and Ti, or another stable substance when intended for permanent use. The drug(s) may also be entrapped into the metal of the stent or graft body, which has been modified to contain micropores or channels. Also lumenal and/or ablumenal coating or external sleeve made of polymer or other biocompatible materials, e.g. as disclosed above, that contain the drug(s) can also be used for local delivery.

Examples of polymeric materials include hydrophilic, hydrophobic or biocompatible biodegradable materials, e.g. polycarboxylic acids; cellulosic polymers; starch; collagen; hyaluronic acid; gelatin; lactonebased polyesters or copolyesters, e.g. polylactide; polyglycolide; polylactide-glycolide; polycaprolactone; polycaprolactone-glycolide; poly(hydroxybutyrate); poly(hydroxyvalerate); polyhydroxy(butyrate-co-valerate); polyglycolide-co-trimethylene carbonate; poly(diaxanone); polyorthoesters; polyanhydrides; polyaminoacids; polysaccharides; polyphospoeters; polyphosphoester-urethane; polycyanoacrylates; polyphosphazenes; poly(ether-ester) copolymers, e.g. PEO-PLLA, fibrin; fibrinogen; or mixtures thereof; and biocompatible non-degrading materials, e.g. polyurethane; polyolefins; polyesters; polyamides; polycaprolactame; polyimide; polyvinyl chloride; polyvinyl methyl ether; polyvinyl alcohol or vinyl alcohol/olefin copolymers, e.g. vinyl alcohol/ethylene copolymers; polyacrylonitrile; polystyrene copolymers of vinyl monomers with olefins, e.g. styrene acrylonitrile copolymers, ethylene methyl methacrylate copolymers; polydimethylsiloxane; poly(ethylene-vinylacetate); acrylate based polymers or coplymers, e.g. polybutylmethacrylate, poly(hydroxyethyl methylmethacrylate); polyvinyl pyrrolidinone; fluorinated polymers such as polytetrafluoethylene; cellulose esters e.g. cellulose acetate, cellulose nitrate or cellulose propionate; or mixtures thereof.

Stents are commonly used as a tubular structure left inside the lumen of a duct or vessel to relieve an obstruction. They may be inserted into the duct lumen in a non-expanded form and are then expanded autonomously (self-expanding stents) or with the aid of a second device in situ, e.g. a catheter-mounted angioplasty balloon which is inflated within the stenosed vessel or body passageway in order to disrupt the obstructions associated with the wall components of the vessel and to obtain an enlarged lumen.

For example, the drug(s) may be incorporated into or affixed to the stent in a number cf ways and utilizing any biocompatible materials; it may be incorporated into e.g. a polymer or a polymeric matrix and sprayed onto the outer surface of the stent. A mixture of the drug(s) and the polymeric material may be prepared in a solvent or a mixture of solvents and applied to the surfaces of the stents also by dip-coating, brush coating and/or dip/spin coating, the solvent (s) being allowed to evaporate to leave a film with entrapped drug(s). In the case of stents where the drug(s) is delivered from micropores, struts or channels, a solution of a polymer may additionally be applied as an outlayer to control the drug(s) release; alternatively, the drug may be comprised in the micropores, struts or channels and the adjunct may be incorporated in the outlayer, or vice versa. The drug may also be affixed in an inner layer of the stent and the adjunct in an outer layer, or vice versa. The drug(s) may also be attached by a covalent bond, e.g. esters, amides or anhydrides, to the stent surface, involving chemical derivatization. The drug(s) may also be incorporated into a biocompatible porous ceramic coating, e.g. a nanoporous ceramic coating.

According to the method of the invention or in the device or system of the invention, the drug(s) may elute passively, actively or under activation, e.g. light-activation. The drug(s) elutes from the polymeric material or the stent over time and enters the surrounding tissue, e.g. up to ca. 1 month to 1 year. The local delivery according to the present invention allows for high concentration of the drug(s) at the disease site with low concentration of circulating compound. The amount of drug(s) used for local delivery applications will vary depending on the compounds used, the condition to be treated and the desired effect. For purposes of the invention, a therapeutically effective amount will be administered. By therapeutically effective amount is intended an amount sufficient to inhibit cellular proliferation and resulting in the prevention and treatment of the disease state. Specifically, for the prevention or treatment of restenosis e.g. after revascularization, or antitumor treatment, local delivery may require less compound than systemic administration.

Combinations of at least two representatives of an ARB, an ACEI and a RI have particularly beneficial effects, especially when used in the treatment or prevention of restenosis in diabetic and non-diabetic patients. For example, an ARB and an ACEI or an RI; an ACEI and an RI; or an ARB, an ACEI and an RI can be combined.

In a further embodiment, the present invention relates to;
A method for preventing or treating macrophage, lymphocyte and/or neutrophil accumulation and/or smooth muscle cell proliferation and migration in hollow tubes such as arteries or veins, or increased cell proliferation or decreased apoptosis or increased matrix deposition in a mammal in need thereof for local administration, comprising administering a therapeutically effective amount of an ARB or an RI or at least two representatives selected from the group consisting of an ARB, an ACEI and an RI, or, in each case, a pharmaceutically acceptable salt thereof.
A method for the treatment of intimal thickening in vessel walls comprising the controlled delivery from any catheter-based device or intraluminal medical device of a therapeutically effective amount of an ARBI or an RI or at least two representatives selected from the group consisting of an ARB, an ACEI and an RI, or, in each case, a pharmaceutically acceptable salt thereof. Preferably the administration or delivery is made using a catheter delivery system, a local injection device, an indwelling device, a stent, a coated stent, a sleeve, a stent-graft, polymeric endoluminal paving or a controlled release matrix.
The use of a drug-eluting or drug-releasing stent according to the present invention, or a drug-delivery vehicle according to the present invention, or a drug delivery device or system according to the present invention for the manufacture of a medicament for local administration, for preventing or treating macrophage, lymphocyte and/or neutrophil accumulation and/or smooth muscle cell proliferation and migration in hollow tubes such as arteries or veins, or increased cell proliferation or decreased apoptosis or increased matrix deposition in a mammal in need thereof.
The use of a drug-eluting or drug-releasing stent according to the present invention, or a drug-delivery vehicle according to the present invention, or a drug delivery device or system according to the present invention for the manufacture of a medicament for the treatment of intimal thickening in vessel walls.

In a preferred embodiment the invention relates to a method or use as described above for the prevention or reduction of vascular access dysfunction in association with the insertion or repair of an indwelling shunt, fistula or catheter, preferably a large bore catheter, into a vein or artery, or actual treatment, in a subject in need thereof.

Preferably the invention relates to the prevention or reduction of vascular access dysfunction in hemodialysis, such as restenosis of the anastamosis of a dialysis access graft.

Preferably the treatment of intimal thickening in vessel walls is stenosis, restenosis, e.g. following revascularization or neovascularization, and/or inflammation and/or thrombosis.

In another embodiment the invention relates to the use of an ARBI or an RI or at least two representatives selected from the group consisting of an ARB, an ACEI and an RI, or, in each case, a pharmaceutically acceptable salt thereof for the manufacture of a drug-eluting or drug-releasing stent, a drug delivery vehicle, drug delivery device or system according to the present invention.

Utility of the drug(s) may be demonstrated in animal test methods as well as in clinic, for example in accordance with the methods hereinafter described.

Surprisingly, it has also been found that compounds of the present invention or a pharmaceutically acceptable salt thereof can be suitably administered in the prevention or reduction of vascular access dysfunction that accompanies the insertion or repair of an indwelling shunt, fistula or catheter in a patient in need thereof.

ARBs, ACEIs or RIs, or, in each case, a pharmaceutically acceptable salt thereof show an unexpected high potency to prevent or eliminate vascular access dysfunction because of its unexpected multifunctional activity, and its activity on different aspects of vascular access dysfunction.

Thus in a further aspect the present invention also relates to;
A pharmaceutical composition for preventing or treating restenosis in diabetic and non-diabetic patients, or for the prevention or reduction of vascular access dysfunction in association with the insertion or repair of an indwelling shunt, fistula or catheter in a subject in need thereof, comprising a compound selected from the group consisting of an ARB, an ACEI and an RI, or, in each case, a pharmaceutically acceptable salt thereof, together with one or more pharmaceutically acceptable diluents or carriers therefore.
The use of a compound selected from the group consisting of an ARB, an ACEI and an RI, or, in each case, a pharmaceutically acceptable salt thereof, for the manufacture of a pharmaceutical for preventing or treating restenosis in diabetic and non-diabetic patients, or for the prevention or reduction of vascular access dysfunction in association with the insertion or repair of an indwelling shunt, fistula or catheter in a subject in need thereof.
A method for the prevention or reduction of vascular access dysfunction in association with the insertion or repair of an indwelling shunt, fistula or catheter into a vein or artery, or actual treatment, in a mammal in need thereof, which comprises administering to the subject an effective amount of a compound selected from the group consisting of an ARB, an ACEI and an RI, or, in each case, a pharmaceutically acceptable salt thereof.
A use, method or composition as described above, for use in conjunction with one or more active co-agents.

A preferred ARB is valsartan, a preferred RI is aliskiren, and a preferred ACEI is benazepril or, in each case, a pharmaceutically acceptable salt thereof.

According to the invention, valsartan, benazepril or aliskiren may be applied as the sole active ingredient or in conjunction with each other in the form of dual or triple combinations such as described above. The present invention relates also to the use of a combination comprising at least two representatives selected from the group consisting of valsartan, benazepril, aliskiren, or, in each case, a pharmaceutically acceptable salt thereof.

Preferred combinations comprise valsartan and aliskiren, or valsartan and benazepril, or aliskiren and benazepril or valsartan and benazepril and aliskiren or, in each case, independently of one another, a pharmaceutically acceptable salt thereof.

Preferably the invention relates to a use, method or composition according to the invention, for use in dialysis patients. Preferably the treatment period commences about 7 days prior to access placement.

Preferably the vascular access dysfunction is selected from vascular access clotting, vascular thrombosis or restenosis. Preferably the vascular access dysfunction is the need for an unclotting procedure. In a preferred aspect the dosage is administered orally. Preferably the subject is selected from a dialysis patient, a cancer patient or a patient receiving total parenteral nutrition.

The doses of aliskiren to be administered to warm-blooded animals, for example human beings, of, for example, approximately 70 kg body weight, especially the doses effective in the inhibition of the enzyme renin, are from approximately 3 mg to approximately 3 g, preferably from approximately 10 mg to approximately 1 g, for example approximately from 20 mg to 600 mg mg, or 20 mg to 200 mg per person per day, divided preferably into 1 to 4 single doses which may, for example, be of the same size. Usually, children receive about half of the adult dose. The dose necessary for each individual can be monitored, for example by measuring the serum concentration of the active ingredient, and adjusted to an optimum level. Single doses comprise, for example, 10, 40 or 100 mg per adult patient For oral doses, preferably from 100 up to 600 mg/day.

Valsartan, will be supplied in the form of suitable dosage unit form, for example, a capsule or tablet, and comprising a therapeutically effective amount, e.g. from about 20 to about 320 mg, of valsartan which may be applied to patients. The application of the active ingredient may occur up to three times a day, starting e.g. with a daily dose of 20 mg or 40 mg of valsartan, increasing via 80 mg daily and further to 160 mg. daily up to 320 mg daily.

Preferably, valsartan is applied twice a day with a dose of 80 mg or 160 mg, respectively, each. Corresponding doses may be taken, for example, in the morning, at mid-day or in the evening. Preferred is b.i.d. administration.

In case of ACE inhibitors, preferred dosage unit forms of ACE inhibitors are, for example, tablets or capsules comprising e.g. from about 5 mg to about 100 mg or 5 mg to about 60 mg, preferably 5 mg, 10 mg, 20 mg or 40 mg, of benazepril.

Further benefits when applying a combination of the present invention are that lower doses of the individual drugs to be combined according to the present invention can be used to reduce the dosage, for example, that the dosages need not only often be smaller but are also applied less frequently, or can be used in order to diminish the incidence of side effects.

This is in accordance with the desires and requirements of the patients to be treated.

Preferably, the jointly therapeutically effective amounts of the active agents according to the combination of the present invention can be administered simultaneously or sequentially in any order, separately or in a fixed combination.

The pharmaceutical combinations according to the present invention as described hereinbefore and hereinafter may be used for simultaneous use or sequential use in any order, for separate use or as a fixed combination.

The pharmaceutical preparations are for enteral, such as oral, and also rectal or parenteral, administration to homeotherms, with the preparations comprising the pharmacological active compound either alone or together with customary pharmaceutical auxiliary substances. For example, the pharmaceutical preparations consist of from about 0.1% to 90%, preferably of from about 1% to about 80%, of the active compound. Pharmaceutical preparations for enteral or parenteral, and also for ocular, administration are, for example, in unit dose forms, such as coated tablets, tablets, capsules or suppositories and also ampoules. These are prepared in a manner that is known per se, for example using conventional mixing, granulation, coating, solubulizing or lyophilizing processes. Thus, pharmaceutical preparations for oral use can be obtained by combining the active compound with solid excipients, if desired granulating a mixture which has been obtained, and, if required or necessary, processing the mixture or granulate into tablets or coated tablet cores after having added suitable auxiliary substances.

The dosage of the active compound can depend on a variety of factors, such as mode of administration, homeothermic species, age and/or individual condition.

Preferred dosages for the active ingredients of the pharmaceutical composition or combination according to the present invention are therapeutically effective dosages, especially those which are commercially available.

The dosage of the active compound can depend on a variety of factors, such as mode of administration, homeothermic species, age and/or individual condition.

The pharmaceutical preparation will be supplied in the form of suitable dosage unit form, for example, a capsule or tablet, and comprising a therapeutically effective amount of active compound, and in case of combination being together with the further component(s) jointly effective.

In the present description, the term “treatment” includes both prophylactic or preventative treatment as well as curative or disease suppressive treatment, including treatment of patients at risk of contracting the disease or injury, or suspected to have contracted the disease or injury as well as ill patients. This term further includes the treatment for the delay of progression of the disease or injury.

The term “curative” as used herein means efficacy in treating ongoing diseases or injuries.

The term “prophylactic” means the prevention of the onset or recurrence of diseases or injuries.

The term “delay of progression” as used herein means administration of the active compound to patients being in a pre-stage or in an early phase of the disease or injury to be treated, in which patients for example a pre-form of the corresponding disease is diagnosed or which patients are in a condition, e.g. during a medical treatment or a condition resulting from an accident, under which it is likely that a corresponding disease will develop.

1. Comparison of the effects of orally delivered vs locally delivered valsartan (ARB) or aliskiren (RI) on early neointimal lesion formation at 9 days versus late neointiomal lesion formation at 21 days in the rat carotid artery balloon injury model

Numerous compounds have been shown to inhibit intimal lesion formation at 2 weeks in the rat ballooned carotid model, while only few compounds prove effective at 4 weeks. The compounds used according to the present invention are tested in the following rat model.

Rats are dosed orally with placebo or valsartan or aliskiren. Daily dosing starts 1-5 days prior to surgery and continues for and additional 28 days. Rat carotid arteries are balloon injured using a method described by Clowes et al. Lab. Invest. 1983; 49; 208-215. BrDU is administered for 24 hours prior to sacrifice. Sacrifice is performed at 9 or 21 days post-balloon injury. Carotid arteries are removed and processed for histologic and morphometric evaluation. In this assay, the ability of the compounds used according to the present invention can be demonstrated to significantly reduce neointimal lesion formation following balloon injury at 9 and 12 days. However, by 21 days the reduction in neotintimal lesion size is no longer statistically significant. Statistical analysis of the histologic data is accomplished using analysis of variance (ANOVA). A P<0.05 is considered statistically significant.

In contrast, when valsartan or aliskiren is administered locally to the adventitia adjacent to the ballooned carotid via an Alzet minipump (containing valsartan or aliskiren suspended in vehicle) that is connected to a catheter implanted into the adventitia.

In contrast, when valsartan or aliskiren is administered locally to the adventitia adjacent to the ballooned carotid (via a catheter implanted into the adventitia that is connected to an Alzet minipump containing valsartan or aliskiren suspended in vehicle). Local delivery is achieved by surrounding the ballooned area of carotid area with a polyethylene cuff. A is connected to an Alzet minipump containing valsartan or aliskiren suspended in vehicle). There is potent inhibition of both early (9 days post-ballooning) and late (21-28 days post-ballooning) neointimal lesions using local delivery.

2. Inhibition of smooth muscle proliferation and inflammatory events at 7 days and restenosis at 28 days in the rabbit iliac stent model

A combined angioplasty and stenting procedure is performed in New Zealand White rabbit iliac arteries. Iliac artery balloon injury is performed by inflating a 3.0×9.0 mm angioplasty balloon in the mid-portion of the artery followed by “pull-back” of the catheter for 1 balloon length. Balloon injury is repeated 2 times, and a 3.0×12 mm stent is deployed at 6 atm for 30 seconds in the iliac artery. Balloon injury and stent placement is then performed on the contralateral iliac artery in the same manner. A post-stent deployment angiogram is performed. All animals are fed standard low-cholesterol rabbit chow, receive oral aspirin 40 mg/day daily as anti-platelet therapy and receive a compound used according to the present invention either dosed orally starting 1-3 days prior to stenting or a compound used according to the present invention that is delivered locally by coating it onto the stents. BrDU is administered for 24 hours prior to sacrifice and at either seven or twenty-eight days after stenting, animals are anesthetized and euthanized and the arterial tree is perfused at 100 mmHg with lactated Ringer's for several minutes, then perfused with 10% formalin at 100 mmHg for 15 minutes. The vascular section between the distal aorta and the proximal femoral arteries is excised and cleaned of periadventitial tissue. The stented section of artery is embedded in plastic and sections are taken from the proximal, middle, and distal portions of each stent. All sections are stained with hematoxylin-eosin and Movat pentachrome stains or special immunohistochemical stains are used to allow identification of macrophages or lymphocytes or sections are specially processed to allow analysis of cell proliferation by quantification of BrDU positive cells. The number of macrophages, lymphocytes or BrDU positive smooth muscle cells is quantitated and/or computerized planimetry is performed to determine the area of the internal elastic lamina (IEL), external elastic lamina (EEL) and lumen. The neointimal area and neointimal thickness is measured both at and between the stent struts. The vessel area is measured as the area within the EEL. Data are expressed as mean±SEM. Statistical analysis of the histologic data is accomplished using analysis of variance (AN OVA) due to the fact that two stented arteries are measured per animal with a mean generated per animal. A P<0.05 is considered statistically significant.

In this model, treatment with a compound used according to the present invention causes areduction in restenotic lesion formation at 7 and 28 days post-stenting. Both mean neointimal thickness and percent stent stenosis was reduced when arteries from valsartan-treated animals were compared with those from placebo-treated animals. In contrast, there is extensive smooth muscle proliferation, macrophage and lymphocyte accumulation and neointimal formation in placebo-treated animals at both 7 and 28 days.

3. Inhibition of macrophage and lymphocyte accumulation and atherosclerosis progression in mouse models of atherosclerosis.

Male or female, 4-6 week old LDL receptor deficient (LDLr−/−) or ApoE deficient (ApoE−/−) mice from Jackson Labs, Bar Harbor, Me., are divided into treatment groups of 18 animals each. All animals are fed a modified western diet containing 21% butter fat & 1.25% cholesterol for up to 19 weeks. At 15 weeks, one group of animals of each strain is sacrificed to serve as pretreatment, baseline controls. The remaining three groups of LDLr−/− or ApoE−/− animals are dosed orally once a day with vehicle or valsartan or aliskirin, from week 15 through week 19 of diet administration. These mice are sacrificed at the end of week 19. At sacrifice for each time point, arterial samples included the entire aorta and its major branches including the innominate/brachiocephalic, right and left carotids, and the left subclavian. Atherosclerosis extent is quantified for both the aorta and innominate arteries. In addition, the number of inflammatory cells (macrophages and lymphocytes) is quantitated within the arterial samples using special immunohistochemical stains. To quantify aortic lesion extent, aorta are pinned out and gross lesion extent, expressed as a percent of aorta covered by lesion, is determined. Innominate and carotid arteries are embedded in parafin, cross-sectioned, and stained with hemotoxylin and eosin, elastin stains or special stains used to identify and quantitate the number of macrophages or lymphocytes. Intimal lesion area is quantified using a computerized image analysis system. Treatment with a compound used according to the present invention reduces both atherosclerotic lesion extent and atherosclerotic lesion progression compared with placebo treatment.

Significant progression of aortic atherosclerosis is observed in both LDLr− and ApoE− mice between weeks 15 and 19 of diet administration. In both LDLr−/− and ApoE mice, treatment with a compound used according to the present invention results in significantly less aortic lesions compared with controls at 19 weeks. Furthermore, aortic lesion progression appeared to have been effectively halted by the treatment with a compound used according to the present invention. In addition, the number of inflammatory cells (macrophages and lymphocytes) is reduced by 40-50% by treatment with a compound used according to the present invention. Similar effects on atherosclerotic lesion formation and inflammatory cell infiltration are observed in the innominate and carotid arteries.

4. Inhibition of angiogenesis and neovascularization in the mouse and rat following Angil infusion and in the rabbit following stenting. CYR61, an angiogenic factor, is induced by Angiotensin II (Ang II) in vascular cells and tissue. Likewise, Ang II induces an angiogenic reponse when Angil is delivered locally in mice or rats. Valsartan or aliskiren show potent inhibition of this angiogenic response in vivo. Since angiogenesis has been shown to be a key mechanism in the development of restenotic lesions following stenting (Farb et al, Circulation 105:2974, 2002) the anti-angiogenic effect of valsartan or aliskiren are involved in the inhibition of restenotic lesion formation in the rabbit stent model described in Section 2. Compared with placebo-treated rabbits valsartan (or aliskiren) administered both orally and locally via diffusion from a valsartan-coated stent (or aliskiren-coated stent) markedly inhibits the angiogenic response at 7 and 28 days post-stenting.

Significant progression of aortic atherosclerosis is observed in both LDLr− and ApoE− mice between weeks 15 and 19 of diet administration. In both LDLr−/− and ApoE mice, treatment with a compound used according to the present invention results in significantly less aortic lesions compared with controls at 19 weeks. Furthermore, aortic lesion progression appeared to have been effectively halted by the treatment with a compound used according to the present invention. In addition, the number of inflammatory cells (macrophages and lymphocytes) is reduced by 40-50% by treatment with a compound used according to the present invention, an effect that is thought to be related to inhibition of neovascularization of the atherosclerotic lesions. Similar effects on atherosclerotic lesion formation and inflammatory cell infiltration are observed in the innominate and carotid arteries and are also thought to be related to effects on neovascularization.

5. The favorable effects of the compounds used according to the present invention can furthermore be demonstrated in a randomized, double-blind multi-center trial for revascularization of single, primary lesions in native coronary arteries. The primary endpoint is in-stent late luminal loss (difference between the minimal luminal diameter immediately after the procedure and the diameter at six months). Secondary endpoints include the percentage of in-stent stenosis of the luminal diameter and the rate of restenosis. After six months, the degree of neointimal proliferation, manifested as the mean late luminal loss in the group treated with a coated stent comprising a compound used according to the present invention versus the placebo group treated with a non-coated stent is determined, e.g. by means of a virtual coronary angioscopy providing a 3-dimensional reconstructed internal view of the coronary system, by means of a conventional catheter-based coronary angiography and/or by means of intracoronary untrasound.

6: A stent can be manufactured from medical 316LS stainless steel and is composed of a series of cylindrically oriented rings aligned along a common longitudinal axis. Each ring consists of 3 connecting bars and 6 expanding elements. The stent is premounted on a delivery system. The active agent or combination of active agents (0.50 mg/ml) optionally together with 2,6-di-tert.4-butyl-4-methylphenol (0.001 mg/ml), is incorporated into a polymer matrix based on a semi-crystalline ethylene-vinyl alcohol copolymer. The stent is coated with this matrix.

7: A stent is weighed and then mounted for coating. While the stent is rotating, a solution of polylactide glycolide, 0.70 mg/ml of aliskiren or of at least two representatives selected from the group consisting of valsartan, benazepril, and aliskiren, or, in each case, a pharmaceutically acceptable salt thereof, dissolved in a mixture of methanol and tetrahydrofuran, is sprayed onto it. The coated stent is removed from the spray and allowed to air-dry. After a final weighing the amount of coating on the stent is determined.

8: Stability of aliskiren or a combination of at least two representatives selected from the group consisting of valsartan, benazepril, and aliskiren in pharmaceutically acceptable polymers at body temperature and their release from polymer coatings.

Four 2 cm pieces of coated stents as described above are placed into 100 mL of phosphate buffer solution (PBS) having a pH of 7.4. Another 4 pieces from each series are placed into 100 mL of polyethylene glycol (PEG)/water solution (40/60 v/v, MW of PEG=400). The stent pieces are incubated at 37° C. in a shaker. The buffer and PEG solutions are changed daily and different assays are performed on the solution to determine the released active compounds concentrations. Such assays can show a stable active compounds release from coated stents for more than 45 days. By the term “stable active compounds release” we mean less than 20% preferably less than 10% of variation of the drug release rate. Controlled release techniques used by the person skilled in the art allow an unexpected easy adaptation of the required active compounds release rate. Thus, by selecting appropriate amounts of reactants in the coating mixture it is possible to easily control the bioeffectiveness of the coated stents. Depending on the kind of coating technology used, the drug may be eluted from coating passively, actively or by light activation.

Release of the active compounds in plasma can also be studied. 1 cm pieces of a coated stent are put into 1 mL of citrated human plasma (from Helena Labs.), which is in lyophilized form and is reconstituted by adding 1 mL of sterile deionized water. Three sets of stent plasma solutions are incubated at 37° C. and the plasma is changed daily. In a separate study, it is shown that the active compounds in human plasma were stable at 37° C. for 72 hours.

Angiotensin receptor, Angiotensin converting enzyme and Renin assays are performed separately with the active compounds released from the last piece of each sample, to determine the remaining activity of the released compounds. The inhibition of Angiotensin receptor, Angiotensin converting enzyme and Renin activity in vitro is measured. Such assays can show that the activity of the active compounds released from stent after 45 days is still 90% of that of the normal activity of the active compounds. These assays can prove the unexpected high stability of our preferred active compounds and combinations in polymer coatings.

9: Examples of Synergic Combinations.

Further experiments similar to that of example 1 can reveal synergic combinations when at least two representatives selected from the group consisting of valsartan, benazepril, and a aliskiren, or, in each case, a pharmaceutically acceptable salt thereof, are used in conjunction.

Data points just spanning the IC50 of the agents alone or in combination are entered into the CalcuSyn program (CalcuSyn, Biosoft, Cambridge UK). This program calculates a non-exclusive combination index (Cl), whose value is indicative of the interaction of the two compounds, where Cl˜1 represents nearly additive effects; 0.85-0.9 indicates a slight synergism and a value below 0.85 indicates synergy.

The combinations especially show a synergistic therapeutic effect, e.g. with regard to slowing down, arresting or reversing arteriosclerosis, thrombosis, vascular access dysfunction, restenosis and/or inflammation diseases, but also in further surprising beneficial effects, e.g. allowing for less side-effects, an improved quality of life and a decreased mortality and morbidity, compared to a monotherapy applying only one of the pharmaceutically active ingredients used in the combination.

10: Efficacy of the invented method for the prevention or reduction of vascular access dysfunction in association with the insertion of an indwelling catheter into the vein of a patient is demonstrated by the following.

One hundred fifty prospective dialysis patients, who undergo successful insertion of an indwelling, large bore catheter (coated according to the present invention), into a vein are selected for study. These patients are divided into two groups, and both groups do not differ significantly with sex, distribution of vascular condition or condition of lesions after insertion. One group (about 50 patients) receives coated catheters (hereinafter identified as group 1), and another group (about 100 patients) receives non-coated. In addition, patients may also be given a calcium antagonist, nitrates, anti-platelet agents, etc. The comparative clinical data collected over the observation period of 6 months demonstrate the efficacy of 3 month use of coated catheters for the prevention or reduction of vascular access dysfunction in patients after catheter insertion.

11: Efficacy of the invented method for the prevention or reduction of vascular access dysfunction in association with the insertion of an indwelling catheter into the vein of a patient is demonstrated by the following.

One hundred fifty prospective dialysis patients, who undergo successful insertion of an indwelling, large bore catheter, into a vein are selected for study. These patients are divided into two groups, and both groups do not differ significantly with sex, distribution of vascular condition or condition of lesions after insertion. One group (about 50 patients) receives aliskiren in a daily dose of 100 mg, and another group (about 100 patients) does not receive aliskiren. In addition, patients may also be given a calcium antagonist, nitrates, anti-platelet agents, ACEi angiotensin converting enzyme inhibitors (preferably benazepril), ARBs agiotensin receptor blockers (preferably valsartan), or statins. These drugs are administered for 3 consecutive months following catheter insertion.

The comparative clinical data collected over the observation period of 6 months demonstrate the efficacy of 3 month aliskiren treatment for the prevention or reduction of vascular access dysfunction in patients after catheter insertion.

12: Efficacy of the invented method for the prevention or reduction of vascular access dysfunction in association with the insertion of an indwelling catheter into the vein of a patient is demonstrated by the methodology as described by Dr. Burnett S. Kelly and Col., (Kidney International Volume 62; Issue 6; Page 2272—December 2002) which is incorporated into the present application by reference.

A method to test the effect of compounds on vascular graft stenosis is also described in: Davies M G, Owens E L, Mason D P, Lea H, Tran P K, Vergel S, Hawkins S A, Hart C E, Clowes A W. Effect of platelet-derived growth factor receptor-α and -β blockade on flow-induced neointimal formation in endothelialized baboon vascular grafts. Circ Res 2000; 86:779-786, which is incorporated into the present application by reference.

1-35. (canceled)

36. A drug-eluting or drug-releasing stent comprising a renin inhibitor of formula 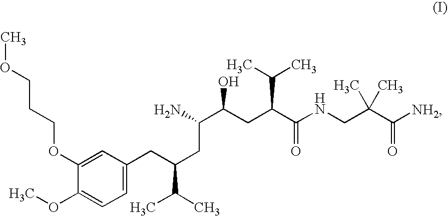
or a pharmaceutically acceptable salt thereof.

37. Stent according to claim 36, comprising an additional compound selected from the group consisting of valsartan and benazepril, or in each case, a pharmaceutically acceptable salt thereof.

38. A drug-delivery vehicle comprising a pharmaceutically acceptable polymer and a compound of formula 1, or a pharmaceutically acceptable salt thereof.

39. Vehicle according to claim 38 wherein the polymer is selected from the group consisting of polyvinyl pyrrolidone/cellulose esters, polyvinyl pyrrolidone/polyurethane, polymethylidene maloeate, polyactide/glycoloide co-polymers, polyethylene glycol co-polymers, polyethylene vinyl alcohol, and polydimethylsiloxane (silicone rubber), also a biocompatible degradable material selected from the group consisting of lactone-based polyesters or copolyesters, polylactide-glycolide; polycaprolactone-glycolide; polyorthoesters; polyanhydrides; polyaminoacids; polysaccharides; polyphosphazenes; poly(ether-ester) copolymers, and a mixture thereof; and biocompatible non-degrading materials, selected from the group consisting of polydimethylsiloxane; poly(ethylene-vinylacetate); acrylate based polymers or copolymers, polybutylmethacrylate, poly(hydroxyethyl methylmethacrylate); polyvinyl pyrrolidinone; fluorinated polymers, polytetrafluoethylene; and cellulose esters.

40. Vehicle according to claim 38, comprising an additional compound selected from the group consisting of valsartan and benazepril, or, in each case, a pharmaceutically acceptable salt thereof.

41. A method for preventing or treating macrophage, lymphocyte and/or neutrophil accumulation and/or smooth muscle cell proliferation and migration in hollow tubes such as arteries or veins, or increased cell proliferation or decreased apoptosis or increased matrix deposition in a mammal in need thereof for local administration, comprising administering a therapeutically effective amount of compound of formula 1, or, a pharmaceutically acceptable salt thereof.

42. A method for the treatment of intimal thickening in vessel walls comprising the controlled delivery from any catheter-based device or intraluminal medical device of a therapeutically effective amount of compound of formula 1, or, a pharmaceutically acceptable salt thereof.

43. A method according to claim 42, wherein the administration or delivery is made using a catheter delivery system, a local injection device, an indwelling device, a stent, a coated stent, a sleeve, a stent-graft, polymeric endoluminal paving or a controlled release matrix.

44. Method according to claim 41, comprising an additional compound selected from the group consisting of valsartan and benazepril or, in each case, a pharmaceutically acceptable salt thereof.

45. A drug delivery device or system comprising a) a medical device adapted for local application or administration in hollow tubes, e.g. a catheter-based delivery device or intraluminal medical device, and b) a compound of formula 1, or, a pharmaceutically acceptable salt thereof,
each being releasably affixed to the catheter-based delivery device or medical device.

46. Device according to claim 45, which is a catheter delivery system, a local injection device, an indwelling device, a stent, a stent-graft or a sleeve.

47. Device according to claim 45, which is a coated stent.

48. Device according to claim 45, comprising an additional compound selected from the group consisting of valsartan and benazepril, or, in each case, a pharmaceutically acceptable salt thereof.

49. A pharmaceutical composition for preventing or treating restenosis in diabetic and non-diabetic patients, or for the prevention or reduction of vascular access dysfunction in association with the insertion or repair of an indwelling shunt, fistula or catheter in a subject in need thereof, comprising a compound of formula 1, or a pharmaceutically acceptable salt thereof, together with one or more pharmaceutically acceptable diluents or carriers therefore.

50. A pharmaceutical composition according to claim 49, comprising an additional compound selected from the group consisting of valsartan and benazepril or, in each case, a pharmaceutically acceptable salt thereof.

51. A method for the prevention or reduction of vascular access dysfunction in association with the insertion or repair of an indwelling shunt, fistula or catheter into a vein or artery, or actual treatment, in a mammal in need thereof, which comprises administering to the subject an effective amount of a compound of formula 1, or a pharmaceutically acceptable salt thereof.

52. The method of claim 51 further comprising one or more active co-agents.

53. The pharmaceutical composition of claim 49 further comprising one or more active co-agents.

54. The method of claim 51, comprising in addition at least one compound selected from the group consisting of valsartan and benazepril or, in each case, a pharmaceutically acceptable salt thereof.

55. The pharmaceutical composition according to claim 49, comprising in addition at least one compound selected from the group consisting of valsartan and benazepril or, in each case, a pharmaceutically acceptable salt thereof.

56. The method of claim 51, for use in dialysis patients.

57. The pharmaceutical composition according to claim 49, for use in dialysis patients.

58. The pharmaceutical composition according to claim 49, wherein the treatment period commences about 7 days prior to access placement.

59. The method of claim 51, wherein the treatment period commences about 7 days prior to access placement.

60. The method of claim 51, wherein the vascular access dysfunction is selected from vascular access clotting, vascular thrombosis or restenosis.

61. The pharmaceutical composition according claim 49, wherein the vascular access dysfunction is selected from vascular access clotting, vascular thrombosis or restenosis.

62. The method of claim 51, wherein the vascular access dysfunction is the need for an unclotting procedure.

63. The pharmaceutical composition according to claim 49, wherein the vascular access dysfunction is the need for an unclotting procedure.

64. The pharmaceutical composition of claim 49, wherein the dosage is administered orally.

65. The method of claim 51 wherein the dosage is administered orally.

66. The pharmaceutical composition claim of 49, wherein the subject is selected from a dialysis patient, a cancer patient or a patient receiving total parenteral nutrition.

67. The method of claim 51, wherein the subject is selected from a dialysis patient, a cancer patient or a patient receiving total parenteral nutrition.

68. The pharmaceutical composition of claim 49, wherein a compound selected from the group consisting of valsartan, benazepril, and a compound of formula 1, or, in each case, a pharmaceutically acceptable salt thereof, is administered.

69. The method of claim 51, wherein a compound selected from the group consisting of valsartan, benazepril, and a compound of formula 1, or, in each case, a pharmaceutically acceptable salt thereof, is administered
